(19) United States
(12) Patent Application Publication
(10) Pub. No.: US 2015/0377901 A1
(43) Pub. Date: Dec. 31, 2015

(54) POINT OF CARE IMMUNIZATION TESTING SYSTEM - DETECTION METHODS

(71) Applicant: ImmunoProfile, LLC, East Brunswick, NJ (US)

(72) Inventor: Sudip Ghatak, East Brunswick, NJ (US)

(21) Appl. No.: 14/749,044

(22) Filed: Jun. 24, 2015

Related U.S. Application Data
(60) Provisional application No. 62/016,975, filed on Jun. 25, 2014.

(51) Int. Cl. G01N 33/68

(52) U.S. Cl. CPC G01N 33/6854; G01N 2570/00; G01N 2469/20

(57) ABSTRACT

A point of care immunization system based upon a range of detection methods to rapidly test a patient in order to ascertain an immunization profile so that vaccinations can be administered to address identified gaps. A point of care system comprised of sample and test strips/cartridges, with said test strips/cartridges configured to meet healthcare requirements of national governing bodies. A point of care system that can function as a stand-alone diagnostic system, stand-alone kit or with a device.

### CROSS-REFERENCE TO RELATED APPLICATION

This application claims benefit of U.S. Provisional Patent Application No. 62/016,975 filed Jun. 25, 2014, which is hereby incorporated in its entirety.

The present invention relates to, among other things, a system to diagnose a patient's immunization status and/or protection levels relative to disease. The invention is comprised of a test strip/cartridge and a means of detecting the signal results from the patients' sample specimen.

The point of care diagnostic instrument measures the antibody levels for various targets based upon a patient's sample. The patient's sample can be blood, saliva, tissue or other bodily fluid or tissue containing antibodies. The targeted test strip/cartridge is configured with the necessary immunization tests per the healthcare guidelines of the specific country for the appropriate group by age, gender, life changing event, and the like [or any other categorization]. The test strip/cartridge may be used with a point of care instrument or the test strip/cartridge may be a self-contained diagnostic device. The system may also update the patient's immunization records and link to the appropriate electronic patient records, laboratory information system, hospital information system and insurance reporting system, among others [or any other information criteria].

The test strip/cartridge may test for a single specific disease or it may contain a complete panel of diseases such that a patient's immunization status and/or protection level may be determined in a single test. The panel of tests may be adjusted from time to time based upon healthcare guidelines and/or the desires of particular clinicians. The test strip/cartridge may be a self-contained unit allowing for a sample specimen to be collected from the patient and delivered in a usable form (dilutions or otherwise) to the test strip/cartridge for analysis and results.

Various types of point of care devices and systems have been proposed. For example, U.S. Prov. Pat. App. 61/563,887 to Ghatak discloses a point of care immunization testing system based on a titration assay comprised of a number of system elements including test kits, diagnostic devices and storage system. For example, U.S. Pat. No. 7,635,594 to Holmes et al. discloses a device for real-time data transmission between a patient and medical practitioners to facilitate high throughput point-of-care testing in detection of disease-indicative analytes including immuno-assays from various bodily fluids.

Point of care diagnostic instruments have been employed for years in medical offices and clinical settings to target various applications. Typically point of care instruments are targeted at rapid testing to detect a patient's exposure to an infectious agent or to provide general information on vital statistics such red blood cell count, white blood cell count and amount of lead present. Infectious disease tests include situations such as determining the presence of streptococcal (strep) bacteria in diagnosing strep throat. Many of these tests fall in the category of CLIA (Clinical Laboratory Improvement Amendments) waived tests, which are defined as simple laboratory examinations and procedures that are cleared by the Food and Drug Administration (FDA) for home use; employ methodologies that are so simple and accurate as to render the likelihood of erroneous results negligible; or pose no reasonable risk of harm to the patient if the test is performed incorrectly.

The main problem is that no point of care device exists to quickly and inexpensively detect a patient's antibody levels to determine immunization protections. Currently, to make such a diagnosis the patient must have blood drawn at a clinical setting and the blood sample tested in a clinical laboratory. The tests are ordered on an individual basis, such as measles, and can take more than a week for response time at high “per test” costs. If a patient needs to be tested for multiple immunization levels, separate and individual tests must be ordered. There is no availability to quickly diagnose a patient's immunity level across the recommended healthcare guidelines in an easy, rapid and cost effective manner. Furthermore, poor record tracking of individual patient's immunizations can result in patients themselves generally not knowing if they are protected.

The range of vaccine efficacies is wide-some as low as 50%, especially in patients with compromised immune systems, and there is no way to know how an individual will respond to the vaccine. For example one of the causes of the 2014 Pertussis (whooping cough) outbreak in the US is due to a low efficacy vaccine. Not only is the duration of protection unpredictable, but other factors can affect a vaccine's efficacy, including missed booster shots, a change in medical condition (HIV, hepatitis, obesity), age of vaccinations, age of vaccines, compromised vaccines, or the like. Certain vaccines cannot be given to children or pregnant women, or while a patient is immuno-compromised. Patients having undergone chemotherapy or radiation therapy may have reduced antibody levels and become immune-compromised.

If a patient is administered an old or even expired vaccine, the patient may not receive full immunity. Recalls occasionally pull out problem vaccine batches, but they are not a guarantee that all vaccines are viable and effective.

Another growing problem is that patients are opting out of vaccines, so communities no longer have near 100% immunization rates. The opt out rate is almost 10% in the US despite the extensive safety record of vaccines. This leads to disease outbreaks in the US and other developed countries when a disease carrier enters a vulnerable population. Disease outbreaks for measles and whooping cough have occurred in the US in 2013 and 2014, along with outbreaks in Europe. The whooping cough outbreak in the US in 2013 reached epidemic rates with incidence in nearly every state. The measles outbreak in the Philippines has exceeded 20,000 cases and a few travelers carried the disease to California which is now experiencing a measles outbreak. Given the ease of travel from vast geographies, a disease carrier can travel almost anywhere in the world in less than twenty-four hours. The only available testing for immunity is costly for the patient, and does not yield immediate results.

A simple but effective solution is needed to enable patients and the healthcare provider the means to quickly test for immunization protection and address any identified gaps through the timely administration of the necessary vaccines. The system needs to be easy to use, timely and targeted, covering a panel of tests. It should comprise of a simple to use diagnostic instrument or self-contained test strip/cartridge. If an instrument is incorporated it should have ports for the sample and the targeted test cartridge. Target test strips/cartridges are for example designed following the recommended guidelines for each country, and are for example adapted for a range of parameters such as age, gender and life changing event or any other criteria. The system may provide timely test results to identify the gaps in the immunization profile and quickly fill the gaps with the timely administration of the necessary vaccine(s). The system may also have a means to update and track patient's immunization records linking it to clinical information systems and providing an electronic/paper copy to the patient.

The system may also serve as a biomarker to indicate the general performance of a person's immune system. For example, if a person has been vaccinated for a number of diseases yet the immune status results show multiple gaps in the profile then it is possible the person may suffer from an underperforming or compromised immune system. Since that is not the expected result from a vaccinated person in generally good health. The person may need further evaluation to determine if they are suffering from an auto-immune disease or any other ailment.

Conventional point of care devices are better suited for disease detection and measure vital parameters from blood and other bodily fluids. Currently no point of care device is targeted for detecting immunization levels leaving most patients uncertain as to how well they are protected against disease until they are exposed to a specific disease threat. This invention makes available point of care testing for both individual disease threats as well as a panel test to determine in a single test the protection levels to multiple diseases.

A number of detection methods are available that can provide superior, more accurate results with greater specificity versus standard titration assay described by Ghatak previously. The results have the potential to be more reliable, sensitive at greater speed. One approach is a more sophisticated titration assay others include using labels, enzymes, ELISA, fluorogenic reporters, electrochemiluminescent tags, label-less assays, sensors, competitive, noncompetitive, heterogeneous and homogeneous assays.

This invention is, in general, to diagnose anti-body levels for immunity levels, using a point of care diagnostic device, targeted test strip/cartridges, detection methods to ascertain results through the employment of a range of signaling mechanisms.

### SUMMARY OF THE INVENTION

The present invention is directed to the diagnosis of antibody or other immunologic component levels in a patient's sample to determine a patient immunization protection levels or allergy indicative IgE levels. (Any reference herein to “immunization testing” includes allergic IgE testing.) The invention is comprised of strips/cartridges, including targeted test strips/cartridges, which may include labels, markings, and controls so that two distinct strips/cartridges cannot be mistaken for each other. The targeted test strip/cartridge can be configured with the necessary immunization tests per the healthcare guidelines of the specific country for the appropriate group by age, gender, life changing event, and the like. The target test strip/cartridge can be self-contained performing all functions on a single test strip/cartridge in a panel test format. The targeted test strip/cartridge can include a smart chip (e.g., transponder) or bar code to instruct the point of care diagnostic device to initiate the appropriate test protocols. In certain embodiments, the invention provides a new and improved point of care testing device to detect antibody levels per immunization protection.

In certain embodiments, the invention provides a biological sample cartridge for ease of use and prevents confusion with the targeted test strip/cartridge. The biological sample strip/cartridge may contain anti-coagulants in the case of blood samples or other specimens to facilitate the sample prep process, enable easier transfer of the blood or other sample and improve assay performance.

In certain embodiments, the invention provides a targeted test strip/cartridge customized to the meet the healthcare requirements for specific countries across age, gender and life changing events among others.

In certain embodiments, the invention provides a method for updating a patient's immunization record for the clinical information systems and patient's personal records.

Provided is an immunization testing device for testing a biological sample from a subject comprising: an analytical module adapted to make one or more dilutions a fluid that is or is derived from the biological sample, contact said dilutions with separate replicates of vaccine- or sensitization-indicative antigens so as to generate a signals indicative of the amount of antigen-reactive immune molecules in the biological fluid dilutions. By “antigen” it is meant any substance (immunologic component) for which immune reactivity is indicative of a subject having immunity to a certain pathogen. As such, an antigen does not need to be directly obtained from the pathogen, as engineered antigens can provide the indicator of immune reactivity.

Provided is an immunization testing device for testing a biological sample from a subject comprising, an analytical module comprising fluidic pathways for conducting dilutions, a controller, a data output device, and one or more input ports having a conjugate input comprising a test cartridge, wherein one or more of the following obtains:

wired connectivity; wireless connectivity; interfaces with hospital/clinical information systems; interfaces with laboratory information systems; provides wired and wireless printer ports; provides links to electronic patient records; provides self-maintenance (e.g., via diagnostic hardware and software for the instrument and/or the cartridges); provides links to smart phones, PDAs printers, and the like; provides biological fluid sample cartridges with:
unique shape, size, color and/or other distinguishable features;
ability to supply needed sample for assay; provides targeted test cartridges with unique shape size, color and/or other distinguishable features and/or ability to supply needed antigens for assay and/or chip to instruct instrument to initiate protocols and/or configured to meet testing requirements as set by healthcare governing bodies and/or individual chambers for each vaccine target with requisite antigen;
   provides smart app with
   smart phones and computer devices; ability to track and update personal immunization records; ability to share personal immunization records
      provides business model for selling vaccines with
   companion diagnostic system for vaccines and/or means for identifying gaps in a person's immunization profile and/or administering only necessary vaccines and/or charging a service or handling fee for vaccine administration and/or charging a fee for the vaccination itself. A method for selling testing systems including test strips/cartridges for use in physician's office or home. A method to diagnose and protect. A method for charging slot fees on a test strip/cartridge for vaccine targets. A method for vaccine inventory management to order vaccines supplies when needed.

Also provided is an immunization testing device for testing a biological sample from a subject comprising, an analytical module comprising a controller, a data output device, and one or more input ports having a conjugate input comprising a test cartridge port, wherein the test cartridge port has an ID receiver for receiving an ID from an immunization test strip/cartridge, wherein the controller is adapted to operate the analytical module to make one or more dilutions of the biological sample, contact said dilutions with separate replicates of vaccine- or sensitization-indicative antigens so as to generate a signals indicative of the amount of antigen-reactive immune molecules in the biological dilutions, interpret the received ID to identify one of a pre-set plurality of available immunization test strips/cartridges, and to utilize the generated signals and the immunization test strips/cartridge to output a report on the immune status of the subject with respect to an array of separate vaccine- or sensitization-indicative antigens or immune components (e.g., 4 or more). The ports can be shaped to accept their conjugate input and not accept the conjugate inputs of other ports present. The biological sample cartridge may or may not contain anti-coagulants in the case of blood or other specimen samples to facilitate the sample prep process, enable easier transfer of the blood or other specimen sample and better assay performance. The first step of the immunoprofile assay is a sample prep step to ensure the blood sample is properly managed in order to serve the purposes of the assay. Part of this effort can be, as needed, to treat the blood sample with anti-coagulant to prevent the blood from clotting which in some instances can have negative effects on the assay process.

In certain embodiments, the controller is adapted to operate with (A) a collection of immunization test strips/cartridges comprising reagents for testing immune status against an array of vaccine- or sensitization-indicative antigens (or immunologic component), the collection including two or more cartridges for testing separate arrays of vaccine- or sensitization-indicative antigens (or immunologic component), the separate arrays adapted for use with separate patient populations, the cartridges having IDs that are distinctive of the separate arrays, wherein the controller reads the ID of a given utilized test strip/cartridge and presents the output report correlating the vaccination status results with the respective vaccine- or sensitization-indicative antigens (or immunologic component) based on the read ID. In one embodiment, the controller is adapted to operate with packs of immunization compositions, separate packs matching the separate arrays of the immunization test cartridges (vaccines matching the vaccine-indicative antigens), the distinct immunization compositions identifiably spatially segregated on the packs, the packs having IDs that are distinctive of the separate arrays of immunization compositions, wherein after presenting an output report the controller compares the ID of a presented pack with the ID of the utilized test cartridge to confirm that the cognate pack has been presented. In one embodiment, the controller is adapted to operate with packs of immunization compositions, separate packs matching the separate arrays of the immunization test cartridges; wherein after presenting an output report the controller compares an ID of a presented immunization composition with the output report data to confirm that the immunization composition matches the tested vaccine-indicative antigens. In another embodiment, the controller further confirms that the presented immunization composition matches a vaccine-indicative antigen (or immunologic component) found to have a deficient immune response.

In certain embodiments, the immunization testing device takes the form of a handheld sampling device, as described below. In certain embodiments, the vaccine indicative antigens are arrayed on a test strip adapted to contact the dilutions by flow of the dilution material through columns of the test strip so as to serially contact the antigens in a panel format with multiple tests. In certain embodiments, the test strips are incorporated into the immunization test cartridges.

Also provided is a collection of immunization test cartridges comprising reagents for testing immune status against an array of vaccine- or sensitization-indicative antigens (or immunologic component), the collection including two or more cartridges for testing separate arrays of vaccine- or sensitization-indicative antigens, the separate arrays adapted for use with separate patient populations, the cartridges having IDs that are distinctive of the separate arrays. Further provided is a kit comprising (A) a collection of immunization test strips/cartridges, and (B) packs of immunization compositions, separate packs matching the separate arrays of the immunization test cartridges, the distinct immunization compositions identifiably spatially segregated on the packs, the packs having IDs that are distinctive of the separate arrays of immunization compositions, the collections and packs adapted to be operative with an analytical module that utilizes the immunization test cartridges to provide subject immune statuses for the antigens of given immunization test cartridges, and which compares the ID of a utilized immunization test cartridge and that of a presented pack to confirm that the cognate pack has been presented.

In certain embodiments, the immunization composition packs comprise a temperature sensor, electronic memory for tracking temperature from the sensor over time, and wherein the packs are adapted to communicate the temperature tracking to the analytical module. In certain embodiments, the immunization test strip/cartridges are in the form of handheld sampling devices.

Further provided is a method of operating the testing device, comprising operating the testing device with a biological sample from a patient utilizing an immunization test cartridge, and thereby outputting a report on the immune status of the subject with respect to an array of separate vaccine- or sensitization-indicative antigens. Also provided is a method of operating the testing device operative with an immunization pack ID reader, comprising operating the testing device with a biological sample from a patient utilizing a said immunization test cartridge, and thereby outputting a report on the immune status of the subject with respect to an array of separate vaccine- or sensitization-indicative antigens; presenting a said immunization pack to the immunization pack ID reader to generate output from the testing device confirming or negating that the immunization pack is the cognate of the test cartridge. The method can further include the controller obtaining from the immunization pack information on immunization stock, calculating the further utilization implied by the immune status report, and, if needed based on this data, generating (i) a report identifying vaccine restocking needs or (ii) a purchase order to a vaccine supplier. The report or purchase order can also be sent by the controller to a user or to the vaccine supplier, such as by email or fax.

Also provided is a method of operating the testing device with an immunization composition ID reader, comprising operating the testing device with a biological sample from a patient utilizing a said immunization test cartridge, and thereby outputting a report on the immune status of the subject with respect to an array of separate vaccine- or sensitization-indicative antigens; presenting a said immunization composition to the immunization composition ID reader to generate output from the testing device confirming or negating that the immunization composition is the immunization composition called for by the immune status report. In certain embodiments of the method, the analytical module is comprised in a handheld sampling device.

The above methods can be conducted at the point-of-care, such as a physician's office, clinic, patient bedside, and the like. The methods can also be applied to home use as well.

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only illustrative embodiments of this invention and are therefore not to be considered limiting of its scope, for the invention may admit to other equally effective embodiments.

These and other features and advantages of embodiments the present invention will be fully apparent from the following description, when taken in connection with the included drawings.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only illustrative embodiments of this invention and are therefore not to be considered limiting of its scope, for the invention may admit to other equally effective embodiments.

FIG. 1 is a detailed view of sample dilution profile.

FIG. 2 is a detailed view of an alternative dilution profile.

FIG. 3 is an overview of a test strip/cartridge.

FIG. 4 is an overview of an alternative test strip/cartridge.

FIG. 5 is a detailed view of a labeled antibody.

FIG. 6 is a detailed view of a labeled antigen.

FIG. 7 is a label-less detection method.

FIG. 8 is a labeled antigen competitive homogeneous assay.

FIG. 9 is a labeled anti-body competitive homogeneous assay.

FIG. 10 is a two-site, noncompetitive assay.

FIG. 11 is alternative two-site, noncompetitive assay.

FIG. 12 is a point of care device with slots for test strip and sample/blood cartridge.

FIG. 13 is a point of care device with slots for test strip, test cartridge and sample/blood cartridge.

FIG. 14 is a point of care device with slot for sample/blood cartridge and multiple test strips/cartridges.

FIG. 15 is a handheld point of care device with sample/blood collection and multiple slots for test strips/cartridges.

FIG. 16 is a CPU circuit layout.

FIG. 17 (A and B) shows to layouts for detection in analytical modules.

FIG. 18 shows an illustrative test strip/cartridge with multiple tests.

FIG. 19 shows an illustrative test strip overlay/mask.

To facilitate understanding, identical reference numerals have been used, where possible, to designate comparable elements that are common to the figures. The figures are not drawn to scale and may be simplified for clarity. It is contemplated that elements and features of one embodiment may be beneficially incorporated in other embodiments without further recitation. For examples, elements and features can be shared between various embodiments that may operate at atmospheric pressure, or higher pressures, depending on among other things the feedstock natural gas pressure available at different locations of the device.

### DETAILED DESCRIPTION OF THE INVENTION

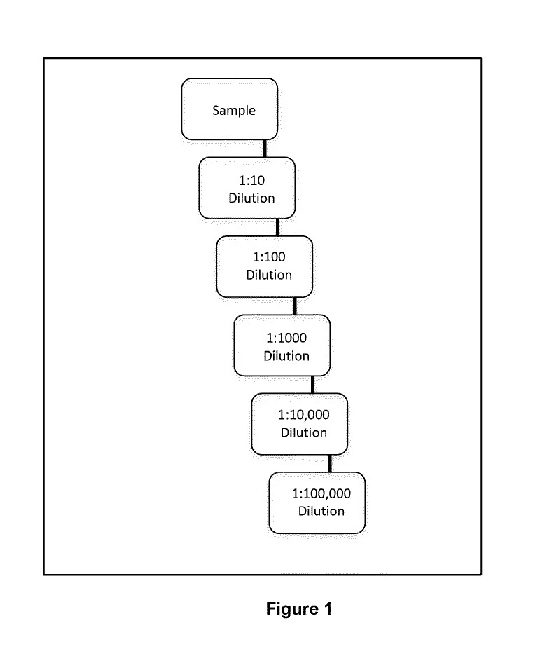

Referring now to the drawings in greater detail, FIG. 1 shows a detailed view of an exemplary sample dilution profile. The sample from the patient will be diluted per the protocols of the test procedure, typically in series fashion. Using fluidic devices (such as microfluidics or robotic pipetting) the sample can be mixed with dilution buffer solution to create the first dilution (e.g. 1:10) which is one part sample and nine parts buffer. The same or alternative dilutions can be conducted on dilutions of a given dilution step. In the Figure we illustrate a simple dilution system, but the point of care device can make virtually any dilution pool by taking the appropriate amount of sample combined with the specified amount of buffer. The figure is only an illustration of one such dilution profile.

The biological sample can be a fluid (e.g., blood, sera, lymph fluid, urine, tears, saliva or the like), a tissue (such as marrow, hair follicles, or the like). In the case of non-fluids, antibodies are extracted. The sample can be from a human, or from any animal with an immune system, such as a dog, cat, horse, donkey, elephant, manatee, etc.

In the diagnostic method conducted in the immunization testing device, dilutions are contacted with the respective vaccine- or sensitization-indicative antigens or immunologic components (or control antigens) and the ancillary agents used to develop a signal indicative of the amount of antibody responsive to the antigen that is present. These ancillary agents can be antibodies, color-developing reagents (inclusive of fluorescence-developing reagents), and the like. Fluorescence can include FRET, wherein fluorescence transfer between two assay moieties enhances signal-to-noise. The diagnostic method (assay) will typically at some point fix the signal on a surface to provide a way of concentrating signal and washing away reagent that might provide a false signal. The vaccine- or sensitization-indicative antigen (or immunologic component) can be provided fixed to the surface, or can be fixed during the course of the diagnostic method. The assay can be a competitive assay, in which a diminution of signal is indicative of unlabeled antibody from the biological sample competing out labeled antibody. Or, the assay can provide a positive correlation between experimental antibody amount and signal, such as via a sandwich assay. In this case, for example, label can be on antigen, on reagent antibody, or the like. Label can be directly measurable, or be the means for developing a measurable signal, such as by being reactive with a binding moiety (e.g., antibody, lectin) having a further label, or by having an attached enzyme.

A given dilution can be assayed against different antigens in separate compartments, or can be assayed such that the signals for the different antigen localize in different places. For example, where the antigen is labeled, it might be captured by different non-competitive antibody localized separately from the capture antibodies for other antigens. Or the antigen (or immunologic component) can be separately localized on a solid. For example, separate, identifiable beads or other solids can be incubated with the dilution to provide separate signals, or signal can be spatially resolved on a surface. Immuno-chromatographic methods (lateral flow) can be used to move reagents over the detection surface and provide washing. In lateral flow, reagents can be provided dry on a portion of a surface, and be solubilized and caused to flow over the signal-generating portion. In certain embodiments, diluted biological can be delivered to an absorbent region, providing a reservoir of fluid for lateral flow.

In certain embodiments, the dilution and/or the presentation and/or removal of reagents for developing signal are delivered robotically, for example using pipetting methods.

In certain embodiments, the dilutions occur on a microfluidics chip or device. This can be referred to as a lab on a chip whereby the dilutions and the corresponding reactions such as a binding assay is performed on the same microfluidics chip with a means to detect the generated signal.

The methods and devices of the invention are adapted to facilitate assaying for a substantial set (e.g., four or more) of vaccination statuses in one relatively quick operation beginning with drawing a biological sample. This set can be termed a “diagnosis set.” Test strips/cartridges are adapted to provide the all of the vaccine- or sensitization-indicative antigens of a given set. Sets are designed to be particularly useful for particular populations. For example, one set is used for preschoolers, another for male teens, another for female teens, another for travelers to a given region, and the like.

The analytical module can contain a controller 50 (FIG. 16), which comprises a central processing unit (CPU) 54, a memory 52, and support circuits 56 for the CPU 54 and is coupled to and controls the various elements of the immunization testing device or, alternatively, operates to do so in conjunction with computers (or controllers) connected to the immunization testing device. For example, another electronic device can supply software, or operations may be calculated off-sight with controller 50 coordinating off-sight operations with the local environment. The controller 50 may be one of any form of general-purpose computer processor that can be used for controlling various devices and sub-processors. The memory, or computer-readable medium, 52 of the CPU 54 may be one or more of readily available memory such as random access memory (RAM), read only memory (ROM), flash memory, floppy disk, hard disk, or any other form of digital storage, local or remote. The support circuits 56 are coupled to the CPU 54 for supporting the processor in a conventional manner. These circuits can include cache, power supplies, clock circuits, input/output circuitry and subsystems, and the like. Methods of operating the immunization testing device may be stored in the memory 52 as software routine that may be executed or invoked to control the operation of the immunization testing device 100. The software routine may also be stored and/or executed by a second CPU (not shown) that is remotely located from the hardware being controlled by the CPU 54. While the above discussion may speak of the “controller” taking certain actions, it will be recognized that it may take such action in conjunction with connected devices. It may or may not depend upon algorithms that are either contained within the devices or accessed externally.

In certain embodiments, the controller is a smart phone, tablet, PC or the like that connects to the analytical module by wire connections or wirelessly.

In certain embodiments certain of the logic circuits or algorithms may be distantly external, such as in Canada. In such embodiments the “controller” is made up of the electronic elements at or near the point-of-care that coordinate data going to such distant logic circuits and operative instructions derived from such distant logic circuits.

In collecting the sample, either via the sample cartridge, test cartridge or a conventional or proprietary collection tube, the collection will generally be labeled with patient identifying information. It may be that the labeling will be via a microchip, RFID, bar code or matrix code (2D bar code) that is rich in information. Thus, a data input device for the immunization testing device can be a scanner for this data rich information.

The immunization testing device may be adapted to operate in conjunction with one or a variety of devices utilized with electronic medical records, such as i-Pads, Android devices, i-Phones, laptops, and the like. In this manner, associating the testing with patient information can be facilitated through automatic input, and data that must be keyboarded in (or voice to text converted) or can be inputted with a device that is better optimized for these functions. Even where significant data input and output is done via an linked device, it can be useful to have an output screen of the device, since after initiation it may prove convenient for the associated medical records device to be used with another patient in another room. More immediate input tools can also be useful, such as just a few buttons whose functions change with the circumstances as announced by for example an adjacent portion of an output screen.

Thus, the output device of the immunization testing device can be a wire output (e.g., USB), or a wireless transmitter (e.g., Bluetooth).

The test strip/cartridge(s) are preferably one test cartridge, but in certain embodiments two or more test cartridges are used. The test strip/cartridge(s) provide the consumable analytical reagents, and may include dilution fluid (e.g., should such not be presented by a biological sample cartridge). In certain embodiments, the test cartridge(s) provide all sample contacting materials, such as disposable pipette tips, or fluidic pathways, or reaction wells, or the like, or all such elements used downstream of biological sample dilution. Inputs (such as a test strip/cartridge) into the analytical module can have a unique shape (such as guide features) such that it can only be connected to its cognate port. Such inputs and the port can be marked with a shared, otherwise unique color or symbol to facilitate quick connection to the correct port.

In certain embodiments, all the reagents, including the vaccine- or sensitization-indicative antigens of a diagnosis set, are provided on one test cartridge. Other test cartridges may be used to provide reagents that do not vary across analyses of diagnosis sets.

In certain embodiments, inputs or other components have IDs, which are symbols, codes (such as linear or multi-dimensional bar codes), devices responsive to electromagnetic queries to emit an electromagnetic identifier (e.g., RFID devices), circuit boards (which may connect via electrical contacts or wirelessly) or the like that can be read by reading devices on the analytical module. An ID receiver can be, among other things, electrical contacts. In this embodiment, the testing device's controller reads the ID(s) and utilizes the information as needed for conducting the analytical processing and reporting. It may be that the same processing steps are conducted for all test cartridges and all vaccine- or sensitization-indicative antigens (or immunologic component), in which case the ID information may inform the report generated, or may inform the interpretation of a positive or negative result for a given vaccine- or sensitization-indicative antigen. (Notwithstanding the use of the singular form, a vaccine- or sensitization-indicative antigen may be a pool of antigens.)

IDs can include or imply a range of definitional information, or information that otherwise aids the controller or the user. For example, the ID for the test cartridge can include information on the target subjects, such as age, gender, geography and life changing events, or the like, or information on the cartridge manufacture date or expiration, or the like. The ID may include or imply the antigens used, there locations, and the like.

The controller can operate upon reading the IDs to relay the identifying information to the operator. For example, an output screen may announce “A Test Cartridge Infant Immunization Set Has Been Installed. Is This Correct?” A validating response may be required, as inputted by touchscreen, fixed buttons, keyboard, or the like.

Fluidics technology, which at smaller scales can be designated microfluidics technology, has existed for many years with numerous applications including clinical diagnostic devices. Examples include the methods and apparatuses of Kellogg et al., U.S. Pat. No. 7,476,361 (Microfluidics devices and methods of diluting samples and reagents), which include methods and apparatus for performing small-scaled analytic and synthetic procedures. The devices and methods utilize centripetal force resulting from rotation of a platform to motivate fluid movement through channels. Serial dilutions are provided. In another example, Schulte et al., US Patent Application No. 2004/0229378 A1 (Well plate microfluidics), discloses in devices and methods for performing a fluidic processes. The Schulte device includes a well plate comprising a plate and an array of wells formed on or in the plate and a fluidic structure connecting at least two of the wells. The device relies on gravitational and capillary forces that exist in channels within the fluidic structure when receiving fluid streams. Methods and devices for moving fluids with electrodes (electrohydrodynamically) and controlling the flow are described for example in U.S. Pat. Nos. 5,992,820, 6,106,685 and 6,109,717. Microfluidic methods using a pumping fluid to indirectly move fluids with electrodes are described in U.S. Pat. No. 5,961,800 These patent disclosures are incorporated herein in there entirety.

Where fluidic controls, such as electrically operated valves or electrodes, reside in a test cartridge, the cartridge can be adapted to contact electrical leads to the analytical module upon attachment of the cartridge to the module. In certain embodiments, the electrical contacts are located above the fluid moving conduits to minimize fluid contact with the electrical components.

The term dilution refers to the reduction in the amount of a particular subject material per unit volume of a fluid containing that material, through the addition of a second fluid or diluents, which dilution can be conducted serially. The diluent may take on a variety of forms, including aqueous and non-aqueous fluids and may include additional material components such as soluble chemical components or suspensions or emulsions of at least partially insoluble components. The subject material composition including chemical compounds either soluble or as suspensions or emulsions, biological material, either soluble or as a suspensions or emulsions and the like. Serial dilution means successive dilutions where the subject material is diluted with diluent to form a first diluted material, which first diluted material is then diluted with a diluent again, to produce a second diluted material, and so on. For example, one produces a first diluted material that is diluted 1:10 over the subject material. By then diluting at least a portion of this material 1:10, one produces a second dilution material that is a 1:100 dilution of the subject material. In general, the methods, devices and systems of the present invention are useful in subject material greater than 10 fold (1:10), typically greater than 100 fold (1:100), preferably greater than 1000 fold (1:1000) and in many cases, greater than 10,000 fold (1:10,000), within a single integrated microfluidic device, which typically has an integral volume, such as channel volume, of less than 10 ul and preferably less than 1 ul. Serial dilutions can also be made in different scales such as an 8 scale whereby the dilutions would be 1:8, 1:64, 1:512 etc. For example, a microfluidic device can inject 1 volume of the subject material and 7 volumes of diluent to a mixing chamber. Mixing can be by magnetic stirrer, ultrasonic, vortexing of the immunization test cartridge, or the like. From there, 7 volumes are available for the assay, and 1 volume can be injected into a next dilution mixing chamber for the next serial dilution.

The ability to perform serial dilutions using different scales can also enable the production of a customized scale such as 1:10, 1:64, 1:100, 1:512, 1:1000 etc, This is accomplished by selecting the appropriate amount of diluted sample and diluting it with the required amount of diluent. Methods and devices for serial dilutions and controlling the flow are described for example in U.S. Pat. Nos. 5,869,004, which is incorporated herein in its entirety.

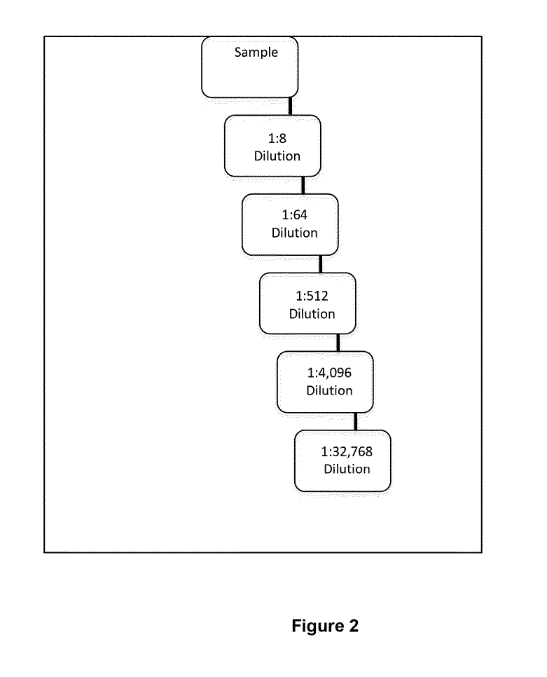

FIG. 2 shows an alternative dilution profile on a 1:8 scale. Other dilution profiles can exist in other numeric increments such as 1:5; 1:6; 1:7 and the like. Different vaccine targets are recommended at different dilution profiles a range of which can be accomplished with microfluidics technology or micro-pipetting. The diluted samples are then injected into the next serial chamber to be combined with the buffer. The point of care testing system whether it is based on microfluidics or micro-pipetting can accomplish different dilution profiles either simultaneously or in sequence to enable the employment of the sample to target which is the labeled, unlabeled, bound or unbound antigen (or immunologic component).

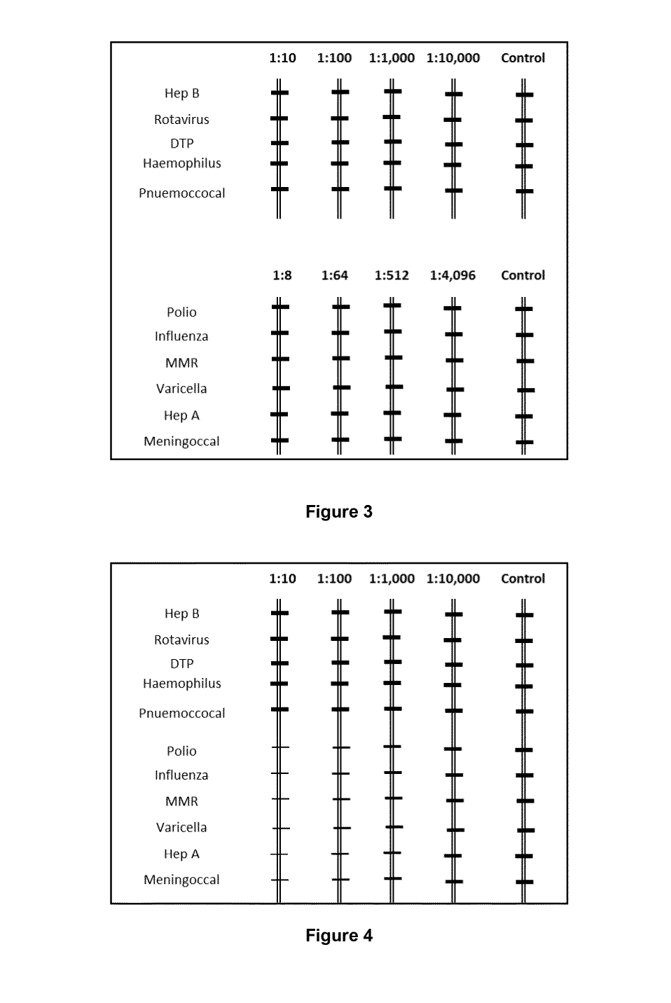

FIG. 3 shows a detailed view of an illustrative diagnostic assay plate, which in certain embodiments can be a test cartridge, or incorporated into a test cartridge. The assay plate can be populated with the prescribed dilution pool samples tested against the target antigens. Each well in the assay plate can be populated with equal amounts of antigen from the targeted test cartridge. For example, in the first column, Hep B antigen is present in each well in equal amounts. In certain embodiments, the Hep B antigen is taken from the target test cartridge and distributed in equal amounts into the designated portion of the assay plate. The sample dilutions are also introduced, generally in equal and specified volumes into each well. A color change for example will occur if the antibody is present in sufficient amount in the diluted sample upon binding with the antigen. Next the instruments analyze the sample dilutions that exhibit a signal change and the ones that do not. This data is then used to deduce a dose response curve. The patient's dose response curve is matched against a standard vaccine titer, such as may be designated by a healthcare agency such as the CDC (Centers for Disease Control). If the dose response curve meets the established standard vaccine titer then no action is needed. If it does not match then the physician may decide to administer a vaccine or vaccine booster per the needs of the patient. This process repeats for each of the antigens in the test cartridge(s). By testing against the recommended panel of target vaccinations a patient will have a complete picture of their immunization profile and how well they are protected against disease. Gaps in the immunization profile can be easily addressed through the administration of the necessary vaccines from the vaccine supply packs.

The embodiment illustrates a test strip/cartridge that can have two separate designations on the same test strip/cartridge to accommodate the different dilution profiles per the recommended vaccine dilution profiles. The test strip/cartridge can have more separate designations for example to accommodate a 1:7 scale dilution profile. The test strip/cartridge can have one or more of these designated areas on the same strip or cartridge as needed.

The test strip/cartridge can also be a microfluidics chip or lab on a chip whereby separate areas on the chip are designated for to test the previously diluted samples in their appropriate areas. The samples come in contact with each chamber that has an equal amount of the target antigen. If the antibody is present in the sample a signal will be emitted that the microfluidics chip can detect then the signals can be read or plotted on a dose response curve to determine if there are any gaps in the patient's immunoprofile.

Biological samples used with the immunization testing device are those that can include antibodies indicative of an immune response in measurable amounts. Often, the biological samples are blood or blood serum, but other biological fluids such as urine or saliva may be used. Biological fluids can be treated to remove larger or higher density elements such as RBCs, lymphocytes or platelets. Such treatment can be by filtration or centrifugation. For blood, an anticoagulant may or may not be used during collection. In certain embodiments, such treatment is done on the immunization testing device with components provided by a biological sample cartridge. Centrifugation can be in-line centrifugation where lower density material flows down the center of a revolving tube. In certain embodiments, there is one biological sample cartridge, which can contain the disposable materials used for pre-dilution sample preparation (if any), dilution buffer, a biological sample acquisition port, sample-handling components used through the dilution stage, and the like. In certain embodiments, the biological sample cartridge is simply a biological sample container, which may connect to plumbing supplied by a test cartridge.

FIG. 4 shows a test strip/cartridge with varying amounts of antigen bound targets in each well to accommodate the different dilution profiles into a single set of dilutions. This reduces the mixing efforts to create different dilution profiles described above and simplifies the design of the test strip/cartridge into a single layout. The layout in FIG. 3 shows multiple layouts for each dilution profile. In this embodiment we use the smaller dilution profile 1:10 and add the necessary amounts of antigens to accommodate the different dilution profiles which are larger such as 1:7, 1:8 etc. One skilled in the art can use different dilution profiles to accomplish the same using a different selection of dilution profiles. This design can also be used for a microfluidics panel or lab on a chip whereby the dilutions, test and results can be performed on a single entity avoiding the need for separate test strip/cartridge and device.

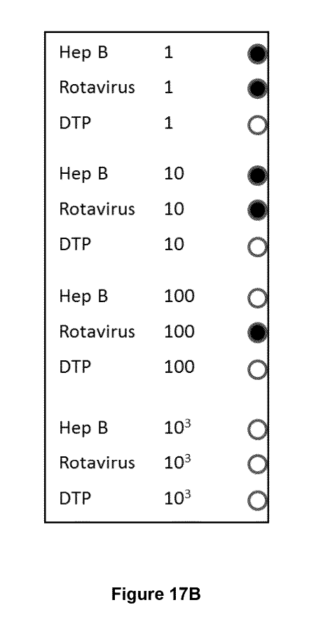

FIG. 17A shows a non-dilution or single dilution assay where, in a given segment, sample is serially passed through progressively higher amounts of the same antigen. As illustrated in FIG. 17A, for Hep B there may be a signal for relative amount 1, a signal for relative amount 10, and none for relative amount 100. The strength of the last positive signal can be calibrated to provide a further refinement of amount. As illustrated in FIG. 17B, the relative amounts do not need to be placed together. Moreover, the relative amounts can be placed in different flow pathways, analogous to FIG. 4, where the dilution indicators 1:10, 1:100, etc., can be replaced with an indicator of relative amount, e.g., 1, 10, 100, etc.

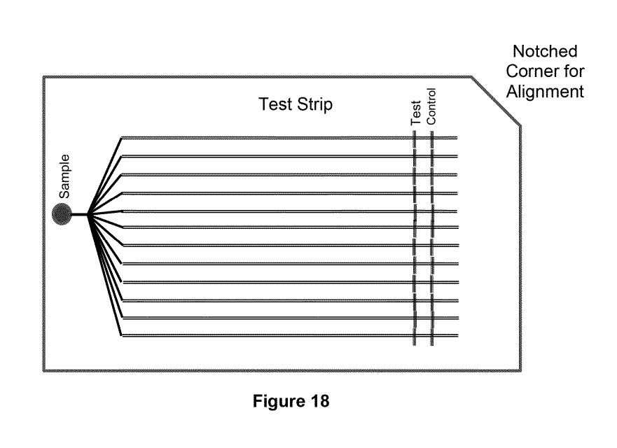

FIG. 18 shows an embodiment with a test strip/cartridge with multiple tests otherwise known as a panel of tests. The sample (blood, saliva, urine etc.) is placed in the chamber for the sample. A sample prep step takes place at the chamber to ensure the sample is properly treated with anti-coagulants and/or other necessary treatments in order to have an accurate test. It is then split 12 ways per the illustration. The test strip/cartridge can be designed for a single test or any number above or below 12 tests. After the sample is split, portions of the sample then migrates down the assay channel. Each assay channel corresponds to an individual immunity test. If the target antibodies are present in the sample it will bind to the target antigen on the test strip/cartridge. If the antibodies are in the appropriate concentration then a reaction will occur and that portion of the test strip/cartridge will change color or provide some discernible indicator that sufficient quantities of the anti-bodies are present. The control portion serves as a reference to ensure the test has been run properly. This process repeats for each of the 12 targets illustrated in the figure. Each immune status assay works independently of each other. There is a notch or some other alignment mechanism shown on the top right of the test strip/cartridge to align with the mask/overlay. The notch or alignment mechanism can appear anywhere on the test strip/cartridge we have shown it in this position for illustrative purposes.

The assays on test strip/cartridge may appear in any sequence we have provided one such example in FIG. 18 for illustrative purposes.

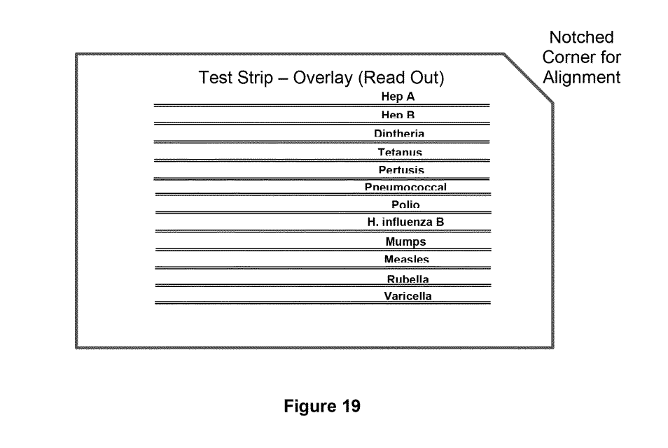

FIG. 19 shows an illustrative test strip overlay/mask. Given the proximity of the lines on the test strip/cartridge it may be advantageous to have an overlay/mask to determine immune status. The overlay/mask aligns with the notch or alignment mechanism on the test strip/cartridge to provide the user a clear indication of which assay is which. The user simply looks for the color change or other indicator for each corresponding immunity test (assay) to determine if the patient is positive (protected) or negative (not protected). The user simply reads the column of color changes (each test can have a different color or other indicator to make the reading process easier) to determine which ones are negative (no color change or other indicator change). If there are the gaps in the person's immunoprofile those can be addressed with targeted vaccinations.

Just one or a very few amounts of a given antigen can be loaded in an analytical module, with the amount calibrated for each given antigen to provide a response that is indicative of immunity or sensitization. In other words, the analytical module does not need necessarily to indicate a positive but low amount if it does not translate to immunity or sensitization, or indicate an amount that is higher than needed to confirm immunity or sensitization.

For assays conducted with non-diluted samples, those of skill will recognize that filtering or centrifugation can be used to remove some or all cells, such as RBCs. Filtering can be incorporated in a flow pathway from the sample application point to the detection point(s).

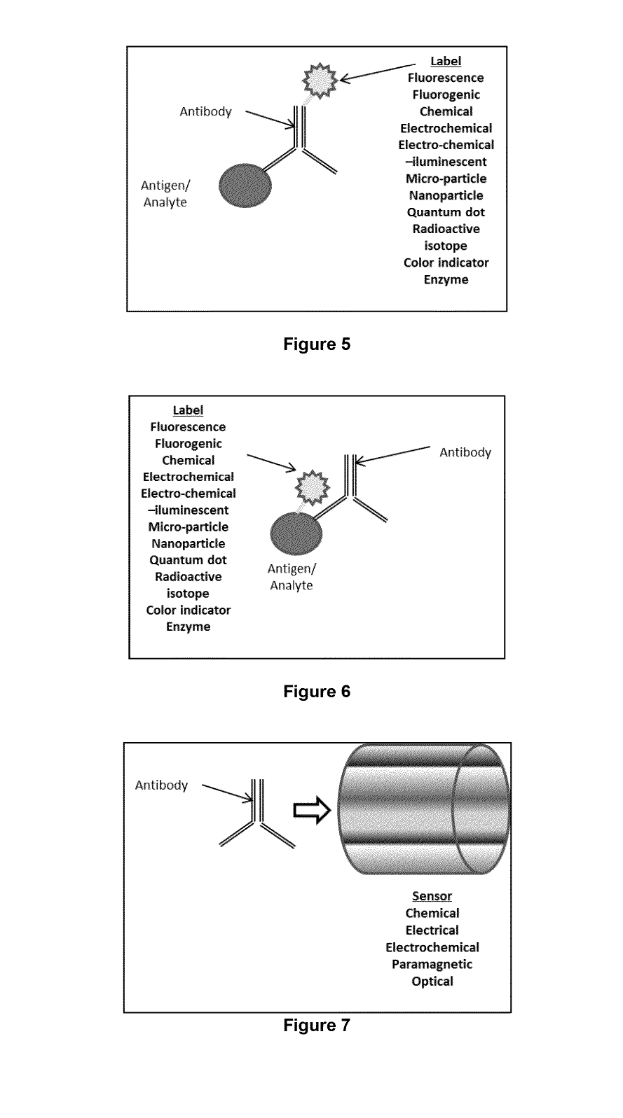

FIG. 5 shows a labeled antibody which is used in traditional immunoassays to find the target antigen. By identifying the target antigen one can determine the presence of disease. Immunoassays employ a variety of labels to allow for detection of antigens. Labels are typically chemically linked or conjugated to the desired antibody as shown in this example. Label can be of a variety of type such as a micro-particle, nanoparticle, quantum dot, enzyme, radioactive isotope, fluorogenic reporter, fluorescence, chemical, electro-chemical, electro-chemiluminescent or color indicator. Where antibody is labeled, a competitive assay is typically conducted.

FIG. 6 shows a labeled antigen which is used to determine a person's immune status per the established targets. The labeled antigen binds to the target antibody to determine if the person is protected against the disease. If the antibody is present in the sample a binding event will occur resulting a signal emittance. This signal can then being detected and results ascertained. The method is to detect the signal from the bound label. Labels are typically chemically linked or conjugated to the desired antibody as shown in this example. Label can be of a variety of type such as a micro-particle, nanoparticle, quantum dot, enzyme, radioactive isotope, fluorogenic reporter, fluorescence, chemical, electro-chemical, electro-chemiluminescent or color indicator.

FIG. 7 shows a label-less antibody assay to determine the presence and level of antibody in the sample. The presence of the target antibody indicates the person is protected against those target diseases. The lack of antibodies across the targets indicates potential gaps that may need to be addressed with vaccinations.

While some kind of label is generally employed in immunoassays, there are certain kinds of assays which do not rely on labels, but instead employ detection methods that don't require the modification or labeling the components of the assay. Surface plasmon resonance is an example of a technique that can detect binding between an unlabeled antibody and antigens. Another label-less immunoassay involves measuring the change in resistance on an electrode as antigen binds to it. FIG. 7 shows other means for the detection of antibodies such as chemical, electrical, electrochemical, paramagnetic and optical. In this embodiment, there can be no dilution or a single dilution, with the sensor being responsive to the scale of interaction of the immune response molecule and the antigen affixed to the sensor. This immune-responsive sensor method is well suited for detecting cellular-based immunity or sensitization. Signals from the immune-responsive sensors are generally electrical, but can be light, RF, or the like signals.

In embodiments, for a given antigen, the structure of an individual detector for that antigen will be identifiable, even if the sensors for a spectrum of antigens are incorporated in one device or micro device.

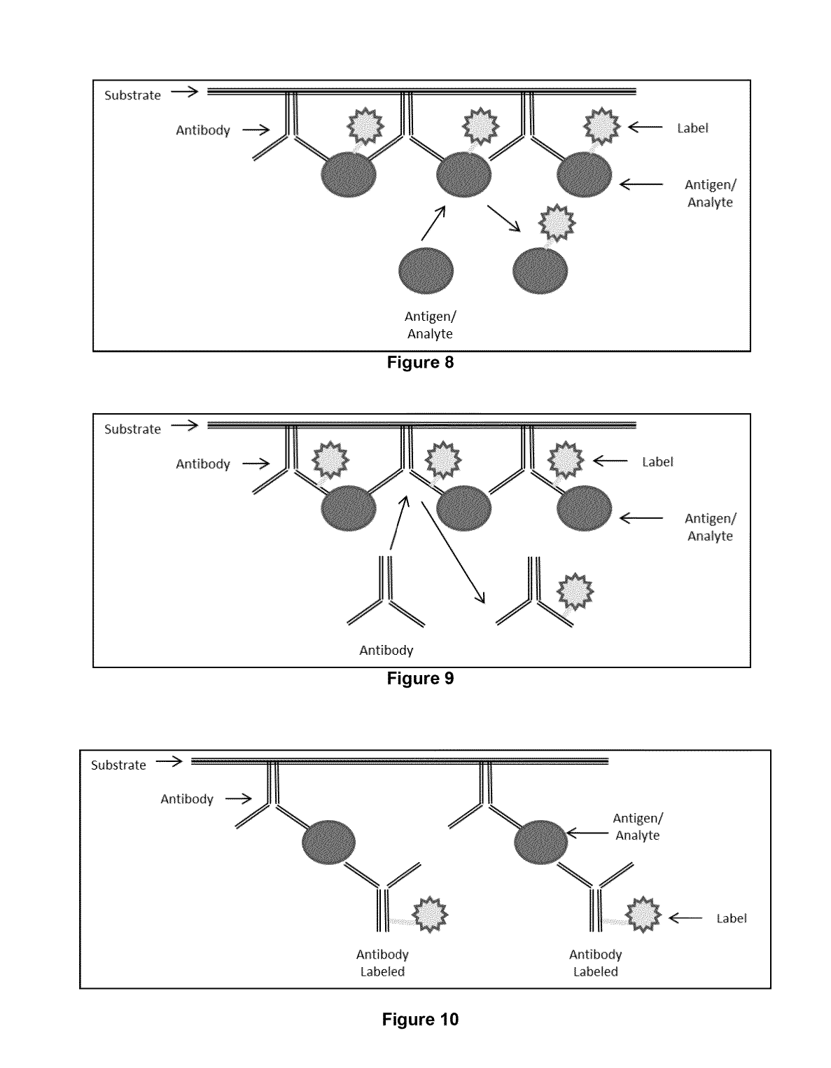

FIG. 8 shows a competitive, homogeneous immunoassay, unlabeled antigen (analyte) in a sample competes with labelled analyte to bind an antibody. The amount of labelled, unbound antigen (analyte) is then measured. In theory the more antigens in the sample, the more labelled antigen gets competed off and hence the amount of labelled, unbound antigen is proportional to the amount of antigen in the sample.

FIG. 9 shows a competitive, homogeneous immunoassay, unlabeled antibody in a sample competes with labelled antibody to bind an antigen. The amount of labelled, unbound antibody is then measured. In theory the more antibodies in the sample, the more labeled antibody gets competed off and hence the amount of labelled, unbound antigen is proportional to the amount of antibody in the sample. This determines the person's immune status levels per each target antibody. If the immune status profile is complete then vaccinations are likely not necessary. If gaps are found then vaccinations maybe required to address them.

FIG. 10 shows a two-site, noncompetitive immunoassay. The antigen in the unknown sample is bound to the antibody site, and then labeled antibody is bound to the antigen. The amount of labeled antibody on the site is then measured. It will be directly proportional to the concentration of the antigen because labeled antibody will not bind if the antigen is not present in the unknown sample. This type is also known as a sandwich assay as the antigen is “sandwiched” between antibodies. ELISA assays are run in this form and used to measure antibody levels. This test is popular in clinical reference labs for its accuracy, sensitivity and speed. It is used to determine a person's immune status levels by determining the antibody levels and identifying any gaps which can be addressed through vaccinations.

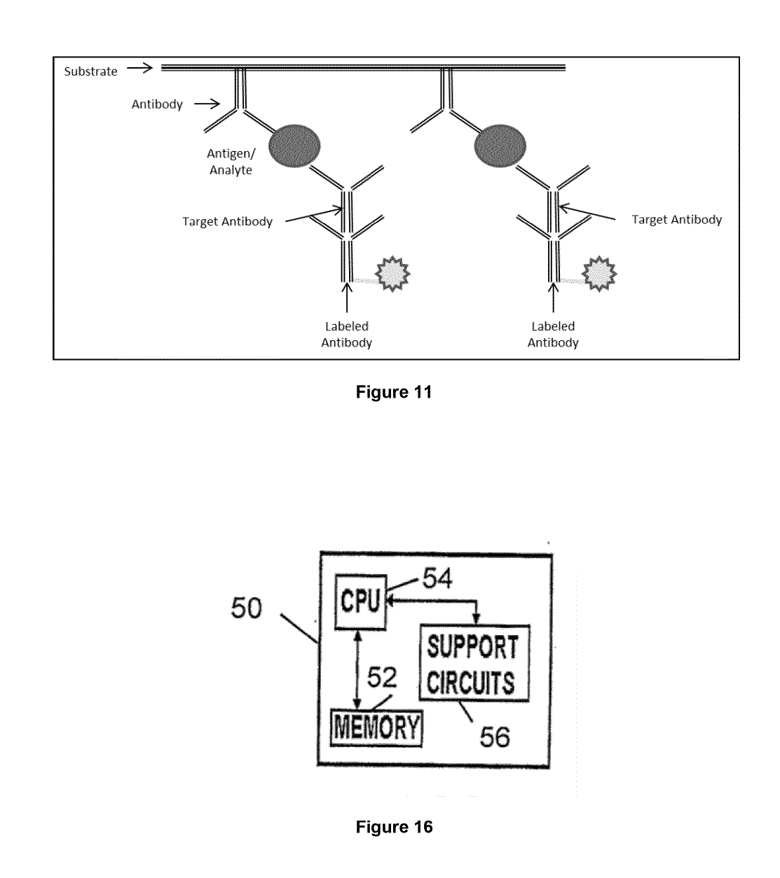

FIG. 11 shows another design of a noncompetitive immunoassay. The target antibody is bound to the antigen and the labeled antibody binds to the target antibody. The assay measures the amount of labeled antibody to determine presence and levels. The results determine whether a person has any gaps in their immune status profile which can be addressed through vaccinations.

As in the competitive, homogeneous assay, in a competitive, heterogeneous immunoassay unlabeled antibodies in a sample compete with labeled antibodies to bind to an antigen. In the heterogeneous assays, the labelled, unbound antibody is separated or washed away; the remaining labelled, bound antibody is measured.

Another assay is the one-site, noncompetitive immunoassay. The unknown antibody in the sample binds with the labeled antigen. The unbound, labelled antigens are washed away, and the bound, labeled antigens are measured, which is directly proportional to the amount of unknown antibody.

Both the competitive, heterogeneous immunoassays and one-site, noncompetitive immunoassays are effective means to determine a person's immune status and any corresponding gaps.

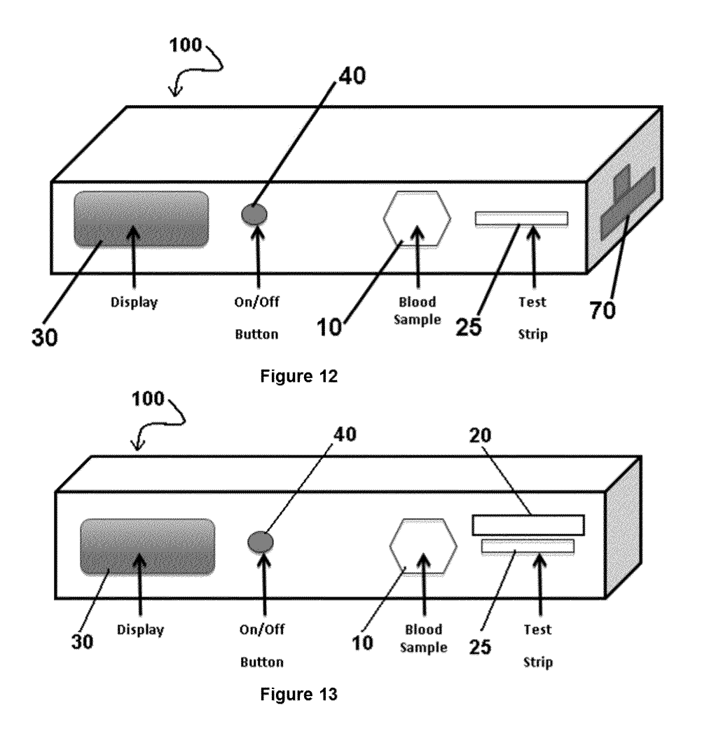

FIG. 12 shows an example of a point of care device with a display, on/off button, slot for sample/blood containing cartridge and slot for test strip. Any of the above mentioned assays can be incorporated into the device/test strip. The design is illustrative and other embodiments may exist.

FIG. 13 shows an example of a point of care device with a display, on/off button, slot for sample/blood containing cartridge, slot for a test cartridge and slot for a test strip. Any of the above mentioned assays can be incorporated into the device and test cartridge/strip.

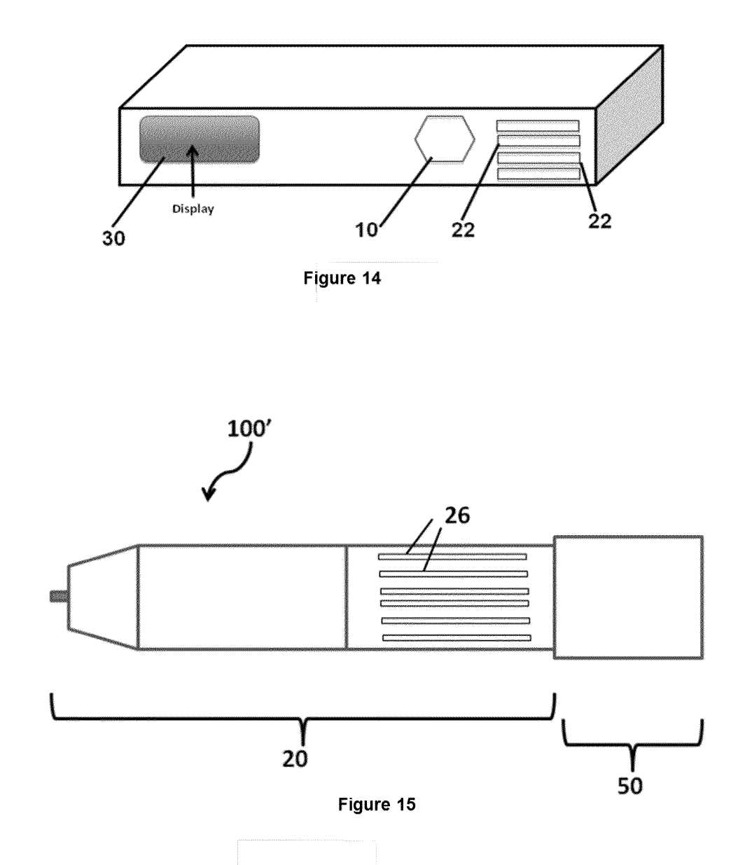

FIG. 14 shows an example of a point of care device with a display, on/off button, slot for sample/blood containing cartridge, multiple slots for test cartridges/strips. Any of the above mentioned assays can be incorporated into the device and test cartridge/strip.

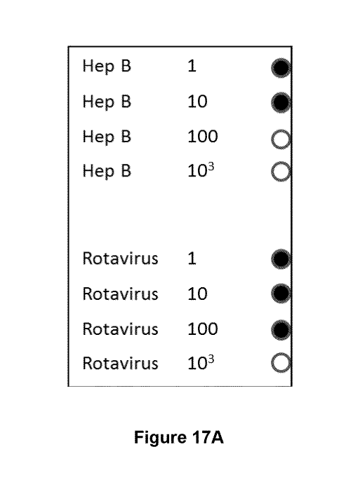

FIG. 15 is an example of a handheld point of care device with ability to collect blood and slots for one or more test cartridge/strip. The person can pierce the skin with an integrated or separate skin piercing tool and then collect the sample into the device. The person can then test against the one or more insert test cartridges/strips. Any of the above mentioned assays can be incorporated into the device and test cartridge/strip.

In certain embodiments, the immunization test cartridge is adapted to provide a sample collection feature. For example, it may incorporate a vacutainer, or be adapted to fit a vacutainer (and for example pierce the septum of the vacutainer to draw biological sample into the test cartridge).

In certain embodiments a sample collection device for blood or saliva is first used to collect the sample. This can be a vacutainer or be adapted to fit a vacutainer. The collection device then integrates into a microfluidics lab on a chip that contains the assay. The entirety of the assay is then performed on the microfluidics lab on chip which contains the entire panel of tests. The microfluidics lab on a chip would be able to perform all the different types of assays mentioned above and have detection capability for appropriate signal emitter employed such as a label. The results of which would then be read by the administrator at the point of care such as a physician's office, clinic or person's home.

The input cartridges can contain circuits that save data on manufacture date or expiration date. Useful circuits include the “Touch Memory” devices from Dallas Semi-Conductors (now a subsidiary of Maxim Integrated Products), which can be adapted to connect by wire or wirelessly to the main instrument upon insertion of the test cartridges. Or, they can contain circuits for monitoring storage conditions, such as temperature. A small power source may be provided on the cartridges to power such monitoring. These circuits are adapted to convey their information to the controller. In certain embodiments, the controller can operate to re-determine an expiration date in view of monitoring data (temperature, humidity, and the like).

Assay detection can be by a single detector (e.g., light absorption detector elements, fluorescence excitation and emission monitoring, and the like) that moves relative to assay sites robotically, is directed to multiple sites with fiber optics, with small-scaled individual detectors, and the like. Or, it can be a CCD or like device that with appropriate lenses monitors all or a useful subset of assay sites simultaneously. Signal can be taken from a liquid phase, or solid phase (assay indicator adsorbed to a surface).

In certain embodiments, the cartridges, such as test strips/cartridges, provide materials for negative and positive controls. Positive controls can be for example control antibody adapted to react with antigen in the positive control assay regions. Negative controls can be antibody that is not matched to a control antigen. The controller can operate to validate or reject a testing run based on the output from the controls, and can be adapted to retrieve trouble-shooting information based on the circumstances of a rejection.

In certain embodiments, the test strips/cartridges contain one or more disease-indicative antigens. These are antigens not supplied in the corresponding vaccine, but which generate antibodies in those exposed to the native causative agent. For example, if a patient is positive for Hep B vaccine antigen and Hep B disease antigen, the physician or clinician can deduce that the patient may or may not have been vaccinated, and has probably been exposed to the virus, in which case further analysis for infection may be in order. These can be termed immune reaction source controls.

As tabulated below, the devices and methods of the invention can in certain embodiments

TABLE

| a. | Provide a point of care instrument; | |
|---|---|---|
| i. | Employ microfluidics for sample and reagent handling; | |
| ii. | Employ micro-titration technology for assays; | |
| iii. | Utilize uniquely (and distinguishably) shaped and/or colored ports; | |
| | a. | Provide a biological sample cartridge |
| | b. | Provide a targeted test cartridge |
| iv. | Utilize a compact, small profile (for the instrument) | |
| v. | Provide rapid testing | |
| vi. | Utilize AC/DC power sources | |
| vii. | Utilize one button operation | |
| viii. | Provide low cost | |
| ix. | Provide durability | |
| x. | Provide wired connectivity | |
| xi. | Provide wireless connectivity | |
| xii. | Interface with hospital/clinical information systems. Interface with | |
| | laboratory information systems | |
| b. | Provide wired and wireless printer ports | |
| xiii. | Provide links to electronic patient records | |
| xiv. | Provide self-maintenance (e.g., via diagnostic hardware and | |
| | software for the instrument and/or the cartridges) | |
| xvii. | Provide links to smart phones, PDAs etc. | |
| xviii. | Provide biological fluid sample cartridges with | |
| | a. | Unique shape |
| | b. | Distinguishable color |
| xix. | Ability to supply needed sample for assay | |
| xx. | Provide targeted test cartridges with | |
| | a. | Unique shape |
| | b. | Distinguishable color |
| | c. | Ability to supply needed antigens for assay |
| | d. | Chip to instruct instrument to initiate protocols |
| | e. | Configured to meet testing requirements as set by healthcare |
| | | governing bodies |
| | f. | Individual chambers for each vaccine target with requisite |
| | | antigen |
| xxi. | Provide vaccine supply pack with | |
| | a. | Compact storage for vaccines |
| | b. | Easy access |
| | c. | Small profile saving space in refrigerator |
| | d. | Smart sensors to indicate temperature |
| | e. | Smart sensors to indicate expiration date |
| | f. | Smart sensors to indicate expiration date |
| | g. | Smart sensors to indicate vaccine supply |
| | h. | Radio frequency tags to establish pedigree |
| | i. | Bar code tags to establish pedigree |
| xxii. | Provide smart app with | |
| | a. | Smart application for smart phones and computer devices |
| | b. | Ability to track and update personal immunization records |
| | c. | Ability to share personal immunization records |
| xxiii. | Provide business model for selling vaccines with | |
| | a. | Companion diagnostic system for vaccines |
| | b. | Means for identifying gaps in a person's immunization profile |
| | c. | Administering only necessary vaccines |
| | d. | Charging a service or handling fee for vaccine administration |
| | e. | Charging a fee for the vaccination itself |

In another embodiment of the invention, the test cartridge incorporates or operates with a test strip (which can be an array of capillaries). The test strip contains the target antigens deposited on the strip. The deposition technology can be printing, lithography, spotting or another method of deposition. The target antigens can be deposited in a series of columns representing different experimental sub-runs, such runs for one or more controls and a number of sample serial dilutions such as 1:10, 1:100, 1:100, etc. Each column has the respective antigen targets such as Hepatitis A, Rotavirus or DTP among other targets (see FIG. 3). The respective diluted sample will be deposited at the top of the column and travel down the designated channel or groove in the test strip. The deposition can contain enough fluid to move the antibodies past all the antigen depositions, or following deposition, sufficient carrier solvent is passed through the strip at the point of sample deposition to move (e.g., by capillary action) the antibodies past all the antigen depositions. The columns of the test strip are fluidically separate such that solvent flowing in one column (e.g., channel) does not carry over to another column.

At each station, the sample encounters a known antigen such as Hepatitis A. If the corresponding antibody is present in the diluted sample a binding event will occur with the target Hepatitis A antigen at the station. The binding reaction would result directly or indirectly in a color, fluorescence, optical density or like change. In certain embodiments, the detectable event is developed by passing developing agents (e.g., labeled anti-human IGG antibodies, enzyme substrates, immunologic components)) down the columns in the same manner as used with the sample, or the like.

The remaining sample will travel to the next station, Rotavirus and if the corresponding antibody is present another binding reaction will take place designated by a direct or indirect detectable change. This will continue for all the remaining antigen stations in each column. If the corresponding antibody is not present in the diluted sample then a binding reaction will not occur and a color change (or the like) will not occur. Once all the reactions have taken place for each antigen station in every diluted sample column a number of stations will have changed color. One then would read the results across each target antigen row such as Hepatitis A and plot the results on a dose response curve per vaccine titer. If the dose response curve per vaccine titer matches the target established by the governing healthcare agency such as the CDC (Centers for Disease Control) in the US, or other similar agencies in other countries, then the patient does not need additional vaccinations. If the dose response curves per vaccine titers do not match then the patient would be advised by their physician to get a vaccination only for the vaccines that are in question. The establishment of the patient's immunity status is thereby called the immunoprofile. Once the necessary vaccines have been updated the patient's immunoprofile can also be updated and disseminated to medical records, patient records and insurance records among others.

A collection of immunization test cartridges comprising reagents for testing immune status against an array of vaccine- or sensitization-indicative antigens, the collection including two or more cartridges for testing separate arrays of vaccine- or sensitization-indicative antigens, the separate arrays adapted for use with separate patient populations, the cartridges having IDs that are distinctive of the separate arrays.

A kit comprising a collection of immunization test cartridges of embodiment L, packs of immunization compositions, separate packs matching the separate arrays of the immunization test cartridges, the distinct immunization compositions identifiably spatially segregated on the packs, the packs having IDs that are distinctive of the separate arrays of immunization compositions, the collections and packs adapted to be operative with an analytical module that utilizes the immunization test cartridges to provide subject immune statuses for the antigens of given immunization test cartridges, and which compares the ID of a utilized immunization test cartridge and that of a presented pack to confirm that the cognate pack has been presented.

The kit wherein the immunization composition packs comprise a temperature sensor, electronic memory for tracking temperature from the sensor over time, and wherein the packs are adapted to communicate the temperature tracking to the analytical module.

The kit wherein the immunization test cartridges are in the form of handheld sampling devices where the method is conducted at the point-of-care.

The Invention can be described further with reference to the following numbered embodiments:

A1. A method for testing a biological sample from a subject for immunization or sensitization status comprising: contacting a biological sample obtained from the subject with one or more types of vaccination- or sensitization-indicative antigens on a test strip or cartridge; and detecting the presence or absence of a threshold amount of one or more antigen-reactive immune mediators present in the biological sample based on the contacting.

A2. The method of one of Embodiment A1, further comprising generating a report on which of the one or more types of vaccination- or sensitization-indicative antigens correspond with a threshold amount of the one or more types of antigen-reactive immune mediators present in the biological sample.

A3. The method of one of Embodiments A1 to A2, wherein the biological sample is an undiluted biological sample.

A4. The method of one of Embodiments A1 to A3, wherein the test strip or cartridge comprises one type of vaccination- or sensitization-indicative antigen.

A5. The method of one of Embodiments A1 to A3, wherein the test strip or cartridge comprises two or more types of vaccination- or sensitization-indicative antigens.

A6. The method of one of Embodiments A1 to A5, wherein the method is performed at a point-of-care.

A7. The method of one of Embodiments A1 to A6, wherein the method is performed at a point-of-assessment.

A8. The method of one of Embodiments A1 to A7, wherein the method is performed at a point-of-diagnosis.

A9. The method of one of Embodiments A1 to A8, wherein the contacting is conducted in one or more immune-responsive sensors, and the detecting comprises signals from the sensors, wherein the signal size for a given vaccination- or sensitization-indicative antigen is indicative of whether the threshold amount is present.

A10. The method of one of Embodiments A1 to A9, wherein the detecting is performed in panel with multiple tests simultaneously or in sequence.

A11. The method of one of Embodiments A1 to A10, wherein the detecting comprises one or more assays selected from the group consisting of: a) a titration assay; (b) a label-based assay; (c) an enzyme assay; (d) an ELISA assay; (e) a fluorogenic reporter assay; (f) an electrochemiluminescent assay; (g) a label-less assay; (h) a sensor-based assay; (i) a competitive assay; (j) a noncompetitive assay; (k) a heterogeneous assay; and (l) a homogeneous assay.

A12. The method of Embodiment A11, wherein the label-based assay utilizes one or more labels selected from the group consisting of: (a) a fluorescence label; (b) a fluorogenic label; (c) a chemical label; (d) an electrochemical label; (e) an electrochemical-luminescent label; (f) a microparticle label; (g) a nanoparticle label; (h) a quantum dot label; (i) a radioactive label; (j) an isotope label; (k) a color indicator label; and (l) an enzyme label.

A13. The method of Embodiment A11, wherein the label-less assay is selected from the group consisting of: (a) surface plasmon resonance; (b) an electrode-based detection assay; and (c) a combination of (a) and (b).

A14. The method of Embodiment A11, wherein the sensor-based assay utilizes one or more sensors selected from the group consisting of: (a) a chemical sensor; (b) an electrical sensor; (c) an electrochemical sensor; (d) a paramagnetic sensor; and (e) an optical sensor.

A15. The method of one of Embodiments A1 to A14, wherein the contacting and detecting is performed on a microfluidics lab-on-a-chip.

A16. The method of one of Embodiments A1 to A14, wherein the contacting and detecting is performed using the test strip or cartridge and a detector or analyzer.

A17. The method of Embodiments A16, wherein the detector or analyzer is a point-of-care detector or point-of-care analyzer.

A18. The method of one of Embodiments A1 to A14, wherein the assay is performed using the test strip or cartridge and a handheld device.

A19. The method of Embodiment A18, wherein the handheld device is a point-of-care handheld device.

A20. The method of one of Embodiments A1 to A19, wherein the biological sample comprises blood, sera, lymph fluid, urine, tears, or saliva.

A21. The method of one of Embodiments A1 to A19, wherein the one or more types of antigen-reactive immune mediators are antibodies.

A22. The method of one of Embodiments A1 to A19, wherein the test strip or cartridge is configured for a specific patient population.

A23. The method of Embodiment A22, wherein the patient population is classified based on age, gender, location, or any combination thereof.

A24. A device for testing a biological sample from a subject for immunization or sensitization status, wherein the device comprises an analytical module adapted to contact an undiluted biological sample with separate replicates of one or more types of vaccine- or sensitization-indicative antigens on a test strip or cartridge configured to generate signals indicative of the amount of one or more types of antigen-reactive immune molecules present in the biological sample.

A25. The device of Embodiment A24, wherein the analytical module comprises: (a) a controller; (b) a data output device, and (c) one or more input ports; wherein the analytical module is adapted to receive an identifier from the immunization test strip or cartridge, and wherein the controller is adapted to operate the analytical module to contact the biological sample with the separate replicates on the test strip or cartridge, to interpret the received identifier to identify one of a pre-set plurality of available test strips or cartridges, and to utilize the generated signals and the identified test strip or cartridge to output a report on the immunization or sensitization status of the subject with respect to the one or more types of vaccine- or sensitization-indicative antigens, and wherein the one or more input ports comprise a conjugate input comprising an immunization test cartridge.

A26. The device of Embodiment A25, wherein any of the one or more input ports are shaped to accept their conjugate input and not accept the conjugate inputs of other ports present.

A27. The device of one of Embodiments A24 to A26, wherein the device is in the form of a handheld sampling device.

A28. The device of one of Embodiments A25 to A27, wherein the controller is adapted to operate with a collection of test strips or cartridges comprising one or more arrays of reagents for testing the immunization or sensitization status of the subject against the one or more types of vaccine- or sensitization-indicative antigens.

A29. The device of Embodiment A28, wherein the collection includes two or more test strips or cartridges for testing separate arrays of the one or more types of vaccine- or sensitization-indicative antigens, wherein the separate arrays are adapted for use with separate patient populations, and wherein the test strips or cartridges have identifiers that are distinctive of the separate arrays.

A30. The device of one of Embodiments A25 to A29, wherein the controller is adapted to operate with packs of immunization compositions, wherein separate packs match the separate arrays of the test strips or cartridges, and wherein the distinct immunization compositions are identifiably spatially segregated on the packs, and wherein the packs have identifiers that are distinctive of the separate arrays of the immunization compositions, and wherein the device comprises an immunization pack identifier reader, and wherein after presenting an output report the controller compares the identifier of a presented pack with the identifier of the test strip or cartridge to confirm that the cognate pack has been presented.

A31. The device of Embodiment A30, wherein the immunization compositions have distinctive identifiers, and wherein the device comprises an immunization composition identifier reader, and wherein after presenting an output report the controller compares an identifier of a presented immunization composition with the output report data to confirm that the immunization composition matches the immunization composition called for by the output report.

A32. The device of one of Embodiments A25 to A31, wherein the controller is adapted to operate with packs of immunization compositions, wherein separate packs match the separate arrays of the test strips or cartridges, and wherein the device comprises an immunization composition identifier reader, and wherein after presenting an output report the controller compares an identifier of a presented immunization composition with the output report data to confirm that the immunization composition matches the tested vaccination- or sensitization-indicative antigens.

A33. The device of Embodiment A32, wherein the controller further confirms that the presented immunization composition matches a vaccination- or sensitization-indicative antigen found to have a deficient immune response.

A34. The device of one of Embodiments A24 to A33, wherein the one or more types of vaccination- or sensitization-indicative antigens are arrayed on a test strip adapted to serially contact the biological sample with the one or more types of vaccination- or sensitization-indicative antigens by flow of the biological sample through columns of the test strip.

A35. The device of Embodiment A34, wherein the test strips are incorporated into cartridges.

B1. A point-of-care method for testing a biological sample from a subject for immunization/sensitization status comprising: without dilution, contacting the sample with a spectrum of vaccination or sensitization-indicative antigens; detecting the presence or absence of a threshold amount of antigen-reactive immune mediators in the sample based on the contacting; and generating a report on which vaccination or sensitization-indicative antigens correspond with a threshold amount of antigen-reactive immune mediators in the sample.

B2. The point-of-care method of Embodiment B1, wherein the contacting is conducted in one or more immune-responsive sensors, and the detecting comprises signals from the sensors whose size, for a given antigen, is indicative of whether the threshold amount is present.

B3. The point-of-care method of one of Embodiments B1 to B2, wherein tests are performed in panel with multiple tests simultaneously or in sequence

B4. The point-of-care method of one of Embodiments B1 to B3, wherein the contacting comprises a titration assay.

B5. The point-of-care method of one of Embodiments B1 to B4, wherein the contacting comprises a label based assay.

B6. The point-of-care method of one of Embodiments B1 to B5, wherein the contacting comprises an enzyme assay.

B7. The point-of-care method of one of Embodiments B1 to B6, wherein the contacting comprises an ELISA assay.

B8. The point-of-care method of one of Embodiments B1 to B7, wherein the contacting comprises a fluorogenic reporter assay.

B9. The point-of-care method of one of Embodiments B1 to B7, wherein the contacting comprises an electrochemiluminescent assay.

B10. The point-of-care method of one of Embodiments B1 to B7, wherein the contacting comprises a label-less assay.

B11. The point-of-care method of one of Embodiments B1 to B7, wherein the contacting comprises a sensor based assay.

B12. The point-of-care method of one of Embodiments B1 to B11, wherein the contacting comprises a competitive assay.

B13. The point-of-care method of one of Embodiments B1 to B11, wherein the contacting comprises a noncompetitive assay.

B14. The point-of-care method of one of Embodiments B1 to B11, wherein the contacting comprises a heterogeneous assay.

B15. The point-of-care method of one of Embodiments B1 to B11, wherein the contacting comprises a homogeneous assay.

B16. The point-of-care method of one of Embodiments B1 to 15, wherein the assay is performed on a microfluidics lab-on-a-chip.

B17. The point-of-care method of one of Embodiments B1 to B15, wherein the assay is performed using a test strip/cartridge and detector/analyzer.

B18. The point-of-care method of one of Embodiments B1 to B15, wherein the assay is performed using a handheld device with test strip(s)/cartridge(s)

B19. A point-of-care method for testing a biological sample from a subject for immunization/sensitization status comprising: contacting the sample with a spectrum of vaccination or sensitization-indicative antigens in one or more immune-responsive sensors; detecting the presence or absence of a threshold amount of antigen-reactive immune mediators in the sample based on the contacting and the size of the signal, for a given antigen, from the sensors; and generating a report on which vaccination or sensitization-indicative antigens correspond with a threshold amount of antigen-reactive immune mediators in the sample.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A method for testing a biological sample from a subject for immunization or sensitization status comprising:
contacting a biological sample obtained from the subject with one or more types of vaccination- or sensitization-indicative antigens on a test strip or cartridge; and
detecting the presence or absence of a threshold amount of one or more antigen-reactive immune mediators present in the biological sample based on the contacting.

2. The method of claim 1, further comprising generating a report on which of the one or more types of vaccination- or sensitization-indicative antigens correspond with a threshold amount of the one or more types of antigen-reactive immune mediators present in the biological sample.

3. The method of claim 1, wherein the biological sample is an undiluted biological sample.

4. The method of claim 1, wherein the test strip or cartridge comprises one type of vaccination- or sensitization-indicative antigen.

5. The method of claim 1, wherein the test strip or cartridge comprises two or more types of vaccination- or sensitization-indicative antigens.

6. The method of claim 1, wherein the method is performed at a point-of-care.

7. The method of claim 1, wherein the method is performed at a point-of-assessment.

8. The method of claim 1, wherein the method is performed at a point-of-diagnosis.

9. The method of claim 1, wherein the contacting is conducted in one or more immune-responsive sensors, and the detecting comprises signals from the sensors, wherein the signal size for a given vaccination- or sensitization-indicative antigen is indicative of whether the threshold amount is present.

10. The method of claim 1, wherein the detecting is performed in panel with multiple tests simultaneously or in sequence.

11. The method of claim 1, wherein the detecting comprises one or more assays selected from the group consisting of:
(a) a titration assay;
(b) a label-based assay;
(c) an enzyme assay;
(d) an ELISA assay;
(e) a fluorogenic reporter assay;
(f) an electrochemiluminescent assay;
(g) a label-less assay;
(h) a sensor-based assay;
(i) a competitive assay;
(j) a noncompetitive assay;
(k) a heterogeneous assay; and
(l) a homogeneous assay.

12. The method of claim 11, wherein the label-based assay utilizes one or more labels selected from the group consisting of:
(a) a fluorescence label;
(b) a fluorogenic label;
(c) a chemical label;
(d) an electrochemical label;
(e) an electrochemical-luminescent label;
(f) a microparticle label;
(g) a nanoparticle label;
(h) a quantum dot label;
(i) a radioactive label;
(j) an isotope label;
(k) a color indicator label; and
(l) an enzyme label.

13. The method of claim 11, wherein the label-less assay is selected from the group consisting of:
(a) surface plasmon resonance;
(b) an electrode-based detection assay; and
(c) a combination of (a) and (b).

14. The method of claim 11, wherein the sensor-based assay utilizes one or more sensors selected from the group consisting of:
(a) a chemical sensor;
(b) an electrical sensor;
(c) an electrochemical sensor;
(d) a paramagnetic sensor; and
(e) an optical sensor.

15. The method of claim 1, wherein the contacting and detecting is performed on a microfluidics lab-on-a-chip.

16. The method of claim 1, wherein the contacting and detecting is performed using the test strip or cartridge and a detector or analyzer.

17. The method of claim 16, wherein the detector or analyzer is a point-of-care detector or point-of-care analyzer.

18. The method of claim 1, wherein the assay is performed using the test strip or cartridge and a handheld device.

19. The method of claim 18, wherein the handheld device is a point-of-care handheld device.

20. The method of claim 1, wherein the biological sample comprises blood, sera, lymph fluid, urine, tears, or saliva.

21. The method of claim 1, wherein the one or more types of antigen-reactive immune mediators are antibodies.

22. The method of claim 1, wherein the test strip or cartridge is configured for a specific patient population.

23. The method of claim 22, wherein the patient population is classified based on age, gender, location, or any combination thereof.

24. A device for testing a biological sample from a subject for immunization or sensitization status, wherein the device comprises an analytical module adapted to contact an undiluted biological sample with separate replicates of one or more types of vaccine- or sensitization-indicative antigens on a test strip or cartridge configured to generate signals indicative of the amount of one or more types of antigen-reactive immune molecules present in the biological sample.

25. The device of claim 24, wherein the analytical module comprises:
(a) a controller;
(b) a data output device, and
(c) one or more input ports;
wherein the analytical module is adapted to receive an identifier from the immunization test strip or cartridge,
and wherein the controller is adapted to operate the analytical module to contact the biological sample with the separate replicates on the test strip or cartridge, to interpret the received identifier to identify one of a pre-set plurality of available test strips or cartridges, and to utilize the generated signals and the identified test strip or cartridge to output a report on the immunization or sensitization status of the subject with respect to the one or more types of vaccine- or sensitization-indicative antigens, and
wherein the one or more input ports comprise a conjugate input comprising an immunization test cartridge.

26. The device of claim 25, wherein any of the one or more input ports are shaped to accept their conjugate input and not accept the conjugate inputs of other ports present.

27. The device of claim 24, wherein the device is in the form of a handheld sampling device.

28. The device of claim 24, wherein the controller is adapted to operate with a collection of test strips or cartridges comprising one or more arrays of reagents for testing the immunization or sensitization status of the subject against the one or more types of vaccine- or sensitization-indicative antigens.

29. The device of claim 28, wherein the collection includes two or more test strips or cartridges for testing separate arrays of the one or more types of vaccine- or sensitization-indicative antigens, wherein the separate arrays are adapted for use with separate patient populations, and wherein the test strips or cartridges have identifiers that are distinctive of the separate arrays.

30. The device of claim 28, wherein the controller is adapted to operate with packs of immunization compositions, wherein separate packs match the separate arrays of the test strips or cartridges, and wherein the distinct immunization compositions are identifiably spatially segregated on the packs, and wherein the packs have identifiers that are distinctive of the separate arrays of the immunization compositions, and wherein the device comprises an immunization pack identifier reader, and wherein after presenting an output report the controller compares the identifier of a presented pack with the identifier of the test strip or cartridge to confirm that the cognate pack has been presented.

31. The device of claim 30, wherein the immunization compositions have distinctive identifiers, and wherein the device comprises an immunization composition identifier reader, and wherein after presenting an output report the controller compares an identifier of a presented immunization composition with the output report data to confirm that the immunization composition matches the immunization composition called for by the output report.

32. The device of claim 28, wherein the controller is adapted to operate with packs of immunization compositions, wherein separate packs match the separate arrays of the test strips or cartridges, and wherein the device comprises an immunization composition identifier reader, and wherein after presenting an output report the controller compares an identifier of a presented immunization composition with the output report data to confirm that the immunization composition matches the tested vaccination- or sensitization-indicative antigens.

33. The device of claim 32, wherein the controller further confirms that the presented immunization composition matches a vaccination- or sensitization-indicative antigen found to have a deficient immune response.

34. The device of claim 24, wherein the one or more types of vaccination- or sensitization-indicative antigens are arrayed on a test strip adapted to serially contact the biological sample with the one or more types of vaccination- or sensitization-indicative antigens by flow of the biological sample through columns of the test strip.

35. The device of claim 34, wherein the test strips are incorporated into cartridges.
